(19) United States
(12) Patent Application Publication
(10) Pub. No.: US 2020/0368142 A1
(43) Pub. Date: Nov. 26, 2020

(54) COSMETIC COMPOSITION FOR IMPROVING SKIN CONDITION COMPRISING MIXTURE OF BRASSICA OLERACEA VAR. ACEPHALA SEAWATER EXTRACT, NASTURTIUM OFFICINALE SEAWATER EXTRACT AND BETA VULGARIS ROOT SEAWATER EXTRACT

(71) Applicant: AMOREPACIFIC CORPORATION, Seoul (KR)

(72) Inventors: Su Jeong SHIN, Yongin-si, Gyeonggi-do (KR); Kil Sun MYOUNG, Yongin-si, Gyeonggi-do (KR); Bo Hyun SHIN, Yongin-si, Gyeonggi-do (KR); Eun Soo LEE, Yongin-si, Gyeonggi-do (KR); Yu jin JIN, Yongin-si, Gyeonggi-do (KR)

(73) Assignee: AMOREPACIFIC CORPORATION, Seoul (KR)

(21) Appl. No.: 16/767,930

(22) PCT Filed: Oct. 26, 2018
(86) PCT No.: PCT/KR2018/012835

§ 371 Date: May 28, 2020

(30) Foreign Application Priority Data
Nov. 29, 2017 (KR) 10-2017-0161705

(51) Int. Cl. A61K 8/9789; A61Q 19/08

(52) U.S. Cl. CPC A61K 8/9789; A61K 2800/74; A61Q 19/08

(57) ABSTRACT

A cosmetic composition containing a mixture of Brassica oleracea var. acephala seawater extract, Nasturtium officinale seawater extract and Beta vulgaris Root seawater extract is disclosed. The cosmetic composition enhances the barrier function of the skin, improves skin moisturizing ability and promotes cell turnover. A use of the composition for improving skin condition and for the manufacture of cosmetics, which function to enhance the barrier function of the skin, improve skin moisturizing ability, and promote skin cell turnover is disclosed.

### TECHNICAL FIELD

One aspect of the present disclosure relates to a cosmetic composition for improving skin condition including a mixture of Brassica oleracea var. acephala seawater extract, Nasturtium officinale seawater extract and Beta vulgaris root seawater extract, and more particularly to a cosmetic composition for enhancing the barrier function of the skin, improving skin moisturizing ability and promoting skin cell turnover, including a mixture of Brassica oleracea var. acephala seawater extract, Nasturtium officinale seawater extract and Beta vulgaris root seawater extract, as an active ingredient

### BACKGROUND ART

The most important function of the epidermis, which is located in the outermost layer of the skin, embraces a protective function of protecting the skin from various external stimuli (physical and chemical stimuli such as chemicals, pollutants, dry environment and UV radiation) and preventing excessive discharge of water in the body through the skin. This protective function can be maintained only when the stratum corneum consisting of keratinocytes is normally formed. The stratum corneum (horny layer), the outermost layer of the epidermis, is formed from keratinocytes and consists of differentiated keratinocytes and lipid layers surrounding them (Elias, J. Invest. Dermatol., 80:44s-49s, 1983). Keratinocytes are characteristic cells formed as basal cells that continuously proliferate in the lowest layer of the epidermis undergo stepwise changes in morphology and functions and thereby move up toward the surface of the skin. After the lapse of a given period, old keratinocytes are detached from the skin and new keratinocytes replace their function, and such a series of repeated change processes is called “differentiation of epidermal cells” or “keratinization”. During the keratinization process, keratinocytes form the stratum corneum, while producing natural moisturizing factors (NMFs) and intercellular lipids (ceramides, cholesterols and fatty acids), providing the stratum corneum firmness and flexibility so as to function as a skin barrier.

However, the stratum corneum can readily lose its functions due to habitual factors such as excessive face washing or bathing, environmental factors such as dry atmosphere or pollutants, and endogenous diseases such as atopic skin or senile skin. In fact, due to various factors which have further increased recently, people suffering from dry skin symptoms and various disorders caused thereby are gradually increasing. Thus, in order to keep the skin moisturized at a suitable level, many studies have been conducted to supply water from outside or prevent loss of water from the body. In fact, various kinds of moisturizers having water holding capability have been developed and used mainly in the cosmetic field.

As harmful factors to the human body gradually increase in the living environment and an aged population increases rapidly, the turnover rate of the stratum corneum be comes slower, and the lipid synthesis capability of keratinocytes is reduced, or the division, growth and differentiation of normal cells in the epidermis become inefficient. As a result, people having skin condition in which the quantity of moisturizing factors and lipids is reduced and thereby the function of the stratum corneum is not maintained, that is, the barrier function of the skin is damaged, is gradually increasing.

Such abnormality in the division and differentiation of epidermal cells causes various skin diseases, including xeroderma, atopic dermatitis and psoriasis, and the symptoms of these skin diseases can be slightly relieved only by the existing moisturizers having water holding capability, but it is difficult to expect fundamental treatment of the diseases.

The human skin undergoes various changes with aging, such as reduced skin thickness, increased wrinkles, decreased elasticity, dull skin complexion, frequent skin troubles, increased liver spots, freckles and age spots, etc.

As the aging progresses, symptoms are exhibited, for example, the content and arrangement of the substances constituting the skin, such as collagen, elastin, hyaluronic acid and glycoprotein, change or decrease, and the skin undergoes oxidative stress by free radicals and reactive oxygen species. It is known that, with the progression of aging or by UV light, the biosynthesis of cyclooxygenase-2 (Cox-2), an enzyme that produces proinflammatory cytokines known to cause inflammation, increases, the biosynthesis of matrix metalloproteinase (MMP), an enzyme that degrades the skin tissue due to these inflammatory factors, increases, and the production of nitric oxide (NO) caused by inducible nitric oxide synthase (iNOS) increases in most cells constituting the skin.

Various cosmetic raw materials having an efficacy of improving skin condition, such as skin moisturization, are being developed. In the case of chemical raw materials, there are problems that side effects including skin allergy or skin trouble may be induced, and in the case of animal raw materials, the use of animal raw materials is rapidly decreasing due to a decrease in consumer preferences and the controversy of mad cow disease. Thus, environment-friendly raw materials such as plant-based raw materials have gained much interest.

Examples of plant-based raw materials may include kale; Brassica oleracea; Brassica oleraceae L. var. acephala DC., watercress; Nasturtium officinale, beet; Beta vulgaris), and the like.

Kale is a second-year herbaceous plant grown from seeds. Kale is very rich in vitamins, minerals, amino acids, fats, proteins, enzymes and fiber. In particular, kale contains up to 405 times the amount of vitamins A, B1, B2, C and calcium (Ca) as other fruits and vegetables, and has a high content of beta-carotene, and thus prevents cancer and lowers cholesterol, thereby preventing the formation of blood clots in the arteries. In addition, since fibers and chlorophylls contained in kale have the efficacy of lowering cholesterol and controlling blood pressure, it is effective in the treatment of lifestyle diseases such as hematopoiesis, anemia, detoxification, gastrointestinal diseases, and the like, and is known to help improve the liver function.

Watercress is a perennial plant native to Central Europe and Central Asia. In Medieval France, essential oils from cresson were used as hair growth agents or hair tonics. Watercress contains high amounts of minerals such as calcium, phosphorus, and iron from a nutritional perspective, and is rich in vitamins A and C, calcium, andiron. It has an effect of preventing oxidation of blood, antifatigue effect, an effect on digestion, and antipyretic effect, and is known to be beneficial for anemia and pregnant women.

Beet is a dicotyledonous annual or biennial plant in the genus of Chenopodium in the order of Centrospermae, and contains galactans, which are polysaccharides, pectin, amino acids such as asparagin and glutamine, and other various ingredients such as organic acids and oligosaccharides. The beetroot has a sugar content of 15 to 16%, the main ingredient of which is sucrose, and is the major source of red pigment. The major pigment of beet is betalain, which contains betacyanin, a red and purple pigment, and betaxanthin, a yellow pigment. The beet has major effects of reducing blood sugar, preventing or alleviating complications in diabetes, treating pain and inflammation, protecting against influenza infection, and has an anti-cancer effect and an anti-viral effect against herpes virus, and is known to have excellent antioxidant power.

It is known that seawater consists of 96.5% by weight of pure water and 3.5% by weight of inorganic salts, and that about 99% of the 30 major elements constituting the inorganic salts are Na⁺, K⁺, Ca²⁺, Mg²⁺, Cl⁻ and SO₄²⁻. The average salt concentration of seawater around the world is about 35‰ (per mil), which means that about 35 g of salt is dissolved in 1 kg of seawater. However, the salt concentration of seawater can vary depending on the amount of solar radiation and evaporation, freshwater inflow due to precipitation, and seawater inflow due to differences in tidal currents. Although the concentration of salt contained in seawater may vary depending on the region, the composition ratio between the major elements of the inorganic salts contained in seawater (Na⁺, K⁺, Ca²⁺, Mg²⁺, Cl⁻ and SO₄²⁻) is relatively constant. Meanwhile, in consideration of the physical properties of seawater, the pH of seawater is pH 8.1, the density thereof is 1.02 to 1.04 g/cm³, the total dissolved solids (TDS) thereof is 35,000 to 45,000 mg/L, and the specific heat, heat capacity and surface tension thereof tend to be large.

Deep ocean water refers to seawater which is 200 m below the sea level, under which the generation of organic matters by photosynthesis does not occur. Since the surface water allows photosynthesis, nitrogen, phosphorus, and nitric acid in water are consumed, but the sunlight does not reach the deep water of 200 m below the sea level, thus, there is no phytoplankton that consumes nutrients, and photosynthesis rarely occurs. Due to this, inorganic nutrient salts such as nitrogen, phosphorus, and nitric acid consumed by photosynthesis remain in the deep water. Since deep ocean water contains large amounts of minerals or essential trace elements necessary for the human body, it can help supplement minerals that tend to be in short supply, and thus can be usefully used in the manufacture of cosmetic compositions.

Magma seawater is water that has been stored for a long time as seawater is naturally filtered through basalt and muddy-sand layers and flowed inside, and is seawater exists at the hybrid zone (interface zone) where the underground freshwater and saltwater meet. In Korea, the magma seawater is distributed in the areas of Jocheon-eup, Namwon-eup and Seongsan-eup in Jeju-do, and its distribution decreases from the coast towards the land. The magma seawater is richer not only in essential minerals, such as sodium, magnesium, calcium, potassium, etc., than regular seawater and deep waters, but also in common useful mineral components, such as iron, manganese, zinc, molybdenum, etc., than deep waters and Samdasoo. In addition, the magma seawater is a clean underground resource without detection of E. coli, nitrate nitrogen, phosphate phosphorus, phenols, etc., and can be usefully used in the manufacture of cosmetic compositions because toxic components, such as arsenic, mercury, cadmium, etc., are not detected or lead is detected in an extremely small amount.

Upon reviewing previous studies on the mixture consisting of kale extract, watercress extract and beetroot extract, it has been reported that the fermented extract of the plant-based mixture containing kale has a skin moisturizing effect, the plant-based mixture containing watercress extract has a hair growth promoting effect, and the beet root extract has a skin moisturizing effect (see, Patent Documents 1 to 3 below). However, no research results that confirmed cosmetically significant activity by applying seawater to kale, watercress, and beetroot have been yet reported.

Under these technical circumstances, the present inventors have made extensive efforts to develop a cosmetic composition for improving skin condition including plant-based extract(s) as an active ingredient, and as a result, confirmed that the composition including a mixture of Brassica oleracea var. acephala seawater extract, Nasturtium officinale seawater extract and Beta vulgaris root seawater extract prepared using seawater shows effects of enhancing the barrier function of the skin and significantly increasing skin moisturizing effect, thereby completing the present disclosure.

### PRIOR ART DOCUMENTS

### Patent Documents

(Patent Document 1) Korean Patent No. 10-0687468 (granted on Feb. 21, 2007)

(Patent Document 2) Korean Patent No. 10-1580283 (Granted on Dec. 18, 2015)

(Patent Document 3) Japanese Unexamined Patent Application Publication No. 2000-290162 (Published on Oct. 17, 2000)

### DETAILED DESCRIPTION OF THE INVENTION

### Technical Problem

It is one object of present disclosure to provide a cosmetic composition for improving skin condition including a mixture of Brassica oleracea var. acephala seawater extract, Nasturtium officinale seawater extract and Beta vulgaris root seawater extract, as an active ingredient.

### Technical Solution

In order to achieve the above object, an aspect of the present disclosure provides a cosmetic composition for improving skin condition including a mixture of Brassica oleracea var. acephala seawater extract, Nasturtium officinale seawater extract and Beta vulgaris root seawater extract, as an active ingredient, and more preferably, a cosmetic composition for enhancing the barrier function of the skin, improving skin moisturizing ability and promoting skin cell turnover including the above-mentioned mixture as an active ingredient.

Another aspect of the present disclosure provides the use of a mixture of Brassica oleracea var. acephala seawater extract, Nasturtium officinale seawater extract and Beta vulgaris root seawater extract in the manufacture of a cosmetic composition or a cosmetic for enhancing the barrier function of the skin, in particular, the use as an enhancer of the barrier function of the skin.

Still another aspect of the present disclosure provides the use of a mixture of Brassica oleracea var. acephala seawater extract, Nasturtium officinale seawater extract and Beta vulgaris root seawater extract in the manufacture of a cosmetic composition or a cosmetic for improving skin moisturizing ability, in particular, the use as an improving agent of skin moisturizing ability.

Yet another aspect of the present disclosure provides the use of a mixture of Brassica oleracea var. acephala seawater extract, Nasturtium officinale seawater extract and Beta vulgaris root seawater extract in the manufacture of a cosmetic composition or a cosmetic for promoting skin cell turnover, in particular, the use as a promoting agent for skin cell turnover.

### Advantageous Effects

The cosmetic composition for improving skin condition including the mixture of Brassica oleracea var. acephala seawater extract, Nasturtium officinale seawater extract and Beta vulgaris root seawater extract according to the present disclosure can be used for the manufacture of cosmetics, which function to enhance the barrier function of the skin, improve skin moisturizing ability and promote skin cell turnover.

### BRIEF DESCRIPTION OF THE DRAWINGS

FIG. 1 shows each of the mRNA expression levels of hyaluronan synthase 2 (HAS2) and filaggrin of keratinocytes upon treatment of the mixture of Brassica oleracea var. acephala extract, Nasturtium officinale extract and Beta vulgaris root extract extracted using seawater or purified water.

FIG. 2 shows the mRNA expression level of occludin (OCLN) of keratinocytes upon treatment of the mixture of Brassica oleracea var. acephala extract, Nasturtium officinale extract and Beta vulgaris root extract extracted using seawater or purified water.

### DETAILED DESCRIPTION OF THE EMBODIMENTS

Unless otherwise defined, all technical and scientific terms used herein have the same meaning as commonly understood by one of ordinary skill in the technical field to which the present disclosure belongs. In general, the nomenclatures used herein are those well-known and commonly used in the art.

The present disclosure will be described in more detail below.

As used herein, the term “about” used to express the length, area, volume, time (duration), concentration, content, temperature, and the like means that there is a maximum tolerance of 10% in the corresponding numerical value or range.

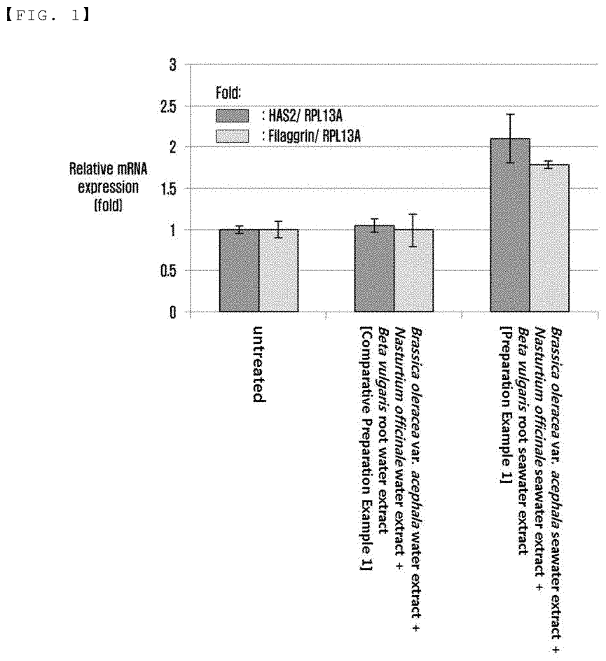

In one embodiment of the present disclosure, the effect of enhancing the skin moisturizing function of the mixture consisting of Brassica oleracea var. acephala seawater extract, Nasturtium officinale seawater extract and Beta vulgaris root seawater extract was confirmed by the mRNA expression levels of hyaluronan synthase 2 (HAS2), which produces hyaluronic acid, a major moisturizing factor for skin, and filaggrin, which is involved in the synthesis of natural moisturizing factors of keratinocytes. As a result, as shown in FIG. 1, Example 1 (mixture consisting of Brassica oleracea var. acephala seawater extract, Nasturtium officinale seawater extract and Beta vulgaris root seawater extract) showed significantly higher mRNA expression levels of HAS2 and filaggrin as compared to Comparative Example 1 (mixture consisting of Brassica oleracea var. acephala water extract, Nasturtium officinale water extract and Beta vulgaris root water extract) (see, Test Example 1).

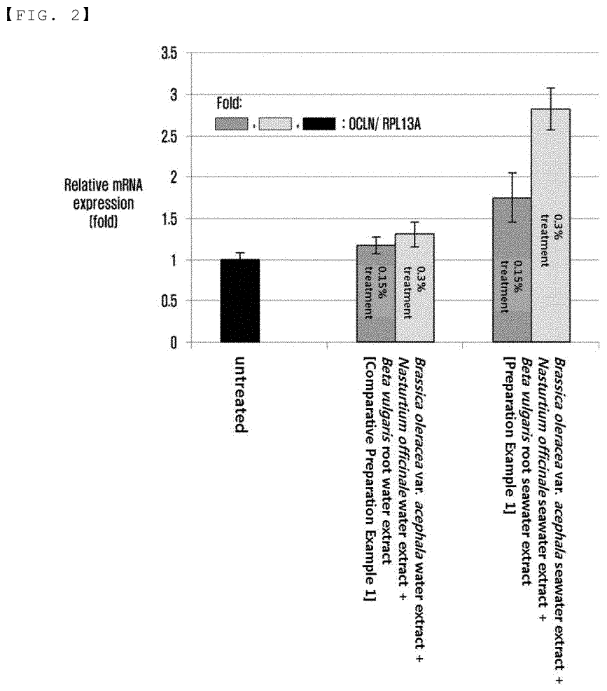

In another embodiment of the present disclosure, the effect of enhancing the barrier function of the skin of the mixture consisting of Brassica oleracea var. acephala seawater extract, Nasturtium officinale seawater extract and Beta vulgaris root seawater extract was confirmed by the mRNA expression level of occludin (OCLN), which is involved in the formation of tight junction (TJ). As a result, as shown in FIG. 2, it was confirmed that (i) Example 1 (mixture consisting of Brassica oleracea var. acephala seawater extract, Nasturtium officinale seawater extract and Beta vulgaris root seawater extract) showed a significantly higher mRNA expression level of OCLN as compared to Comparative Example 1 (mixture consisting of Brassica oleracea var. acephala water extract, Nasturtium officinale water extract and Beta vulgaris root water extract) and (ii) in the case of Example 1, the mRNA expression level of OCLN was significantly higher at the treatment concentration of 0.3% (v/v) as compared to the treatment concentration of 0.15% (v/v) (see, Test Example 2).

Accordingly, in one aspect, the present disclosure relates to a cosmetic composition for improving skin condition including a mixture of Brassica oleracea var. acephala seawater extract, Nasturtium officinale seawater extract and Beta vulgaris root seawater extract as an active ingredient, and more preferably to a cosmetic composition for enhancing the barrier function of the skin, improving skin moisturizing ability and promoting skin cell turnover including the mixture as an active ingredient.

In the present disclosure, the mixture of Brassica oleracea var. acephala seawater extract, Nasturtium officinale seawater extract and Beta vulgaris root seawater extract may be a mixture prepared by obtaining each of seawater extracts from Brassica oleracea var. acephala, Nasturtium officinale and Beta vulgaris root, followed by mixing them, or may be a mixture prepared by mixing Brassica oleracea var. acephala, Nasturtium officinale and Beta vulgaris root, followed by extracting the mixture with seawater. It may preferably be a mixture prepared by obtaining each of seawater extracts from Brassica oleracea var. acephala, Nasturtium officinale and Beta vulgaris root, followed by mixing them.

The Brassica oleracea var. acephala, Nasturtium officinale and Beta vulgaris root may be extracted by a known extraction method for natural substances, and the solvent used for extraction may be seawater. The amount of solvent may vary depending on the amount of Brassica oleracea var. acephala seawater extract, Nasturtium officinale seawater extract and Beta vulgaris Root seawater extract. For example, the seawater extract may be prepared by adding seawater 5 to 1000 times, preferably 10 to 100 times, more preferably 15 to 50 times, and most preferably 20 to 30 times the weight of each of Brassica oleracea var. acephala, Nasturtium officinale and Beta vulgaris root and immersing them therein, followed by mixing each of the seawater extracts in an appropriate ratio. Alternatively, the mixed seawater extract may be prepared by mixing two or three of Brassica oleracea var. acephala, Nasturtium officinale and Beta vulgaris root in an appropriate mixing ratio (weight ratio), followed by adding seawater 5 to 1000 times, preferably 10 to 100 times, more preferably 15 to 50 times, and most preferably 20 to 30 times the weight of the mixture and immersing the mixture therein.

The immersion temperature at the time of preparing the Brassica oleracea var. acephala seawater extract, Nasturtium officinale seawater extract and Beta vulgaris Root seawater extract is 5 to 60° C., preferably 12 to 30° C., and most preferably 10 to 20° C.

The immersion time at the time of preparing the Brassica oleracea var. acephala seawater extract, Nasturtium officinale seawater extract and Beta vulgaris Root seawater extract is not particularly limited, but the immersion time may be, for example, 1 to 24 hours, preferably 2 to 12 hours, and most preferably 4 to 6 hours.

The Brassica oleracea var. acephala seawater extract, Nasturtium officinale seawater extract and Beta vulgaris root seawater extract may be prepared by a known extraction method for natural substances if necessary. For example, it may be extracted by cold water extraction, hot water extraction, ultrasonic extraction, reflux extraction, and heat extraction, and it may be preferably extracted by hot water extraction or reflux extraction, for 1 to 10 times, preferably 2 to 7 times repeatedly.

The Brassica oleracea var. acephala seawater extract, Nasturtium officinale seawater extract and Beta vulgaris root seawater extract may be used as a liquid by filtration, and preferably, it may be used as a solid through a drying process such as spray drying or freeze drying. More preferably, it may be mixed with dextrin before carrying out spray drying or freeze drying and dried.

In the present disclosure, the Brassica oleracea var. acephala seawater extract, Nasturtium officinale seawater extract and Beta vulgaris root seawater extract may be prepared by mixing Brassica oleracea var. acephala seawater extract, Nasturtium officinale seawater extract and Beta vulgaris root seawater extract in a composition ratio of 1:1:3 to 50, more preferably 1:1:4 to 20, and most preferably 1:1:5 to 15 based on the weight.

In the present disclosure, the composition may contain the mixture of Brassica oleracea var. acephala seawater extract, Nasturtium officinale seawater extract and Beta vulgaris root seawater extract in an amount of 0.001 to 40% by weight, more preferably 0.01 to 30% by weight, and most preferably 0.1 to 10% by weight based on the total weight of the composition.

In the present disclosure, in consideration of the formulation stability and the limited content of use based on the regulations concerning the safety of cosmetics, when the total weight of the mixture of Brassica oleracea var. acephala seawater extract, Nasturtium officinale seawater extract and Beta vulgaris root seawater extract is less than 0.001% by weight based on the total weight of the composition, the desired efficacies of enhancing the barrier function of the skin, improving skin moisturizing ability and promoting dead skin cell turnover may be not exhibited as a cosmetic material. In contrast, when the total weight of the mixture of Brassica oleracea var. acephala seawater extract, Nasturtium officinale seawater extract and Beta vulgaris root seawater extract exceeds 40% by weight based on the total weight of the composition, the mixture may not be sufficiently dissolved in the cosmetic formulation, so that it may not be stably mixed with other ingredients of the cosmetic formulation. In addition, there may be problems in terms of safety such as excessive skin irritation when applied to the skin.

In the present disclosure, the seawater may be regular seawater, deep ocean water, magma seawater, etc., but is not limited thereto, and it may be preferably deep ocean water, magma seawater or a mixture thereof. The deep ocean water may be preferably withdrawn at 600 to 660 m under the ocean floor at the offshore, and may vary depending on the characteristics of seawater at the zone where the water is withdrawn. The magma seawater may be preferably withdrawn at 30 to 150 m below the average sea level, preferably 44.35 m, 86.35 m, or 126.35 m by excavating from the ground surface of Suwon region where the magma seawater is gushed out.

In the present disclosure, the density of the seawater may vary depending on the temperature, pressure and salinity of the seawater, but is usually 1000 to 2000 g/L, preferably 1002 to 1100 g/L, and most preferably 1005 to 1045 g/L.

In the present disclosure, the seawater may be seawater desalinated using a conventionally known desalination method, for example, flash vaporization method, seawater freezing method, reverse osmosis, ion exchange resin method, electrodialysis method (ED), or using a commercially available electrodialyzer or desalter after removing impurities such as microorganisms of the seawater collected by a sterile filter, preferably a 0.2 μm filter, and preferably, the seawater desalinated using an electrodialysis method may be desirable as the mineral concentration and hardness of the desalinated seawater can be controlled by varying its electrical conductivity value. For example, the desalinated seawater may be prepared through a desalination process which removes monovalent cations and divalent anions by adjusting the electrical conductivity values to 8 mS/cm, 10 mS/cm, 12 mS/cm, 15 mS/cm, etc.

In the present disclosure, the composition may increase the expression level of a gene encoding hyaluronan synthase 2 (HAS2) of skin cells.

In the present disclosure, the composition may increase the expression level of a gene encoding filaggrin of skin cells.

In the present disclosure, the composition may increase the expression level of a gene encoding occludin (OCLN) of skin cells.

In the composition according to the present disclosure, the seawater may be used not only as an extraction solvent, but also may be contained as an adjuvant for improving the solubility of the mixture of Brassica oleracea var. acephala seawater extract, Nasturtium officinale seawater extract and Beta vulgaris root seawater extract and for further improving the desired effect of improving skin condition.

In the present disclosure, the cosmetic composition including the mixture of Brassica oleracea var. acephala seawater extract, Nasturtium officinale seawater extract and Beta vulgaris root seawater extract may be formulated into any one selected from the group consisting of softening cosmetic water, astringent cosmetic water, nourishing cosmetic water, lotion, eye cream, nourishing cream, massage cream, cleansing cream, cleansing foam, cleansing water, powder, essence, and facial pack.

In addition, the composition may contain adjuvants commonly used in the cosmetic or dermatological field, such as moisturizers (polyols, etc.), viscosity modifiers, thickeners, pH adjusters, fatty materials, organic solvents, solubilizer, concentrating agents, gelling agents, softeners, skin moisturizers, suspending agents, stabilizers, foaming agents, aromatics, surfactants, water, ionic emulsifiers, non-ionic emulsifiers, fillers, sequestering agents, chelators, preservatives, vitamins, humectants, oils, dyes, pigments, hydrophilic activators, lipophilic activator, lipid vesicles, or any other components commonly used in cosmetics. These adjuvants are introduced in amounts commonly used in the cosmetic or dermatological field. In addition, the composition of the present disclosure may contain a skin absorption-promoting substance in order to increase the effect of improving skin condition.

As used herein, the ‘polyol’ included in the composition may mean a polyhydric alcohol, that is, an aliphatic compound having two or more hydroxyl groups (—OH). The one having two hydroxyl groups is called glycol or diol, the one having three hydroxyl groups is called glycerol, and the one having four hydroxyl groups is called pentaerythritol.

According to a preferred embodiment of the present disclosure, the polyol may be selected from the group consisting of sorbitol, polyethylene glycol, polypropylene glycol, dipropylene glycol, propylene glycol, 1,3-butylene glycol, glycerin (glycerol), propanediol, ethyl hexanediol, 1,2-hexanediol, PEG/PPG/polybutylene glycol-8/5/3 glycerin and pentylene glycol, more preferably selected from the group consisting of glycerin, propylene glycol, ethyl hexanediol, propanediol, 1,3-butylene glycol, sorbitol and polyethylene glycol, and most preferably selected from the group consisting of glycerin, propylene glycol, butylene glycol, dipropylene glycol and polyethylene glycol.

The polyol is contained in an amount of 1 to 20% by weight. When the content of the polyol is less than 1% by weight, the emulsifying ability and moisturizing ability may be reduced, and when the content of the polyol exceeds 20% by weight, the feeling of use may be deteriorated.

The oil included in the composition may be at least one selected from the group consisting of hydrocarbon-based oil, higher fatty acid-based oil, ester-based oil and glyceride-based oil, but is not limited thereto.

The oil may be, for example, at least one selected from the group consisting of hydrocarbon-based oil selected from squalane, polybutene, polyisobutene, polydecene, and hydrogenated polydecene; higher fatty acid oils selected from cetyl alcohol, stearyl alcohol, behenyl alcohol, 2-octyldodecanol, and isocetyl alcohol; ester-based oils selected from isopropyl palmitate, 2-octyldodecyl myristate, isopropyl myristate, butyl octyl salicylate, cetyl octanoate, cetyloctyl hexanoate, coco caprylate/caprate, decyl cocoate, isostearyl isostearate, pentaerythrityl tetraethyl hexanoate, and dicaprylyl carbonate; and glyceride-based oils selected from caprylic/capric triglyceride, etc.

The oil may be contained in an amount of 5 to 10% by weight based on the total weight, and when the content is less than or greater than the above range, the stability of the composition may be reduced so that it may be difficult to manufacture various cosmetics products.

In the present disclosure, the viscosity modifier plays a role in further increasing the effect of providing feelings of tension, adhesion and tautness in the skin, thereby increasing the viscosity of the cosmetics, so that the cosmetics can be easily applied.

The viscosity modifier included in the composition may be, for example, at least one compound selected from the group consisting of polyvinyl alcohol, hydroxyethyl acrylate-sodium acryloyldimethyltaurate copolymer, carboxy vinyl polymer, pectin, sodium alginate, carboxymethyl cellulose, hydroxyethyl cellulose, polyacrylic acid, alkyl acrylic acid-acrylic acid copolymer and xanthan gum, but is not limited thereto.

The thickener included in the composition means a substance that increases viscosity, and among them, an oil thickener means a thickener having oil as a main component. The oil thickener may include those based on sucrose esters, alkyl rosinates, dextrin esters, or polyglycerylesters. More specifically, the oil thickener may include at least one selected from the group consisting of dimer dilinoleyl hydrogenated rosinate, phytosteryl/isostearyl/cetyl/stearyl/behenyl dimer dilinoleate, polyglyceryl-10-dodecabehenate, sucrose oleate, sucrose tetrastearate triacetate, glyceryl/polyglyceryl-6-isostearate/behenate esters, dextrin palmitate/ethylhexanoate, dextrin myristate, glycerylbehenate/eicosadioate, and hydrogenated rosin.

Among the chelators included in the composition, disodium EDTA is combined with metal ions and functions to prevent them from catalyzing a catalytic action, and also plays a role in preventing quality deterioration and rancidity of cosmetics, etc., by blocking activities of components used in the manufacture of cosmetics and metal ions that are likely to be mixed in the manufacturing process. In addition, it serves as an accelerator that helps active ingredients to be effectively absorbed into the skin. The disodium EDTA is preferably contained in the range of 0.01 to 0.5% by weight in the cosmetic composition.

Hereinafter, the present disclosure will be described in furthermore detail byway of Example. However, these Examples are given for illustrative purposes only, and it will be obvious to one of ordinary skilled in the art that the scope of the present disclosure is not limited by these examples.

<Preparation of Mixture of Brassica oleracea Var. Acephala Seawater Extract, Nasturtium officinale Seawater Extract and Beta vulgaris Root Seawater Extract>

Raw Materials:

Brassica oleracea var. acephala, Nasturtium officinale and Beta vulgaris root were obtained from retailers, and seawater was supplied from Durae Corporation and used for the preparation of each of Brassica oleracea var. acephala, Nasturtium officinale and Beta vulgaris root extracts.

In order to compare the components of seawater and freshwater, the seawater was subjected to component analysis by the Korea Testing and Research Institute. The mineral contents (unit: ppm) of seawater obtained at different sea levels are shown in Table 1 below.

TABLE 1

| | Seawater | Seawater | Seawater | |
|---|---|---|---|---|
| | 1 (taken at a | 2 (taken at a | 3 (taken at a | |
| Test | depth of | depth of: | depth of: | Fresh |
| Items | 44.35 m) | 86.35 m) | 126.35 m) | water |
| Na | 11400 | 11400 | 10700 | 146 |
| Mg | 1510 | 1520 | 1390 | 0.49 |
| Ca | 460 | 490 | 431 | 9.20 |
| K | 643 | 540 | 621 | 38.5 |
| Cu | 0.00157 | 0.00113 | 0.00080 | 0.00069 |
| Co | — | — | — | — |
| Sn | — | — | — | — |
| Mo | 0.011 | 0.012 | 0.011 | 0.0014 |
| V | 3.28 | 3.53 | 0.01 | 0.013 |
| Ge | 0.0032 | 0.003 | 0.0025 | 0.00019 |
| Br | — | — | — | — |
| Sr | 1.18 | 1.31 | 1.30 | 0.05 |
| Ti | — | — | — | — |
| Ni | — | — | — | — |
| Si (SiO₂) | 7.23 | 6.19 | 1.21 | 1.21 |
| Zn | 0.0045 | 0.0039 | 0.0019 | 0.001 |
| Fe | 0.21 | 0.26 | 0.04 | 0.082 |
| Mn | 19.51 | 20.7 | 18.9 | 0.01 |
| Cl | 16629 | 18500 | 19900 | 262 |
| B | 3.35 | 3.73 | 0.15 | 0.15 |
| Li | 0.37 | 0.41 | 0.09 | 0.09 |
| Ba | 0.032 | 0.019 | 0.015 | 0.015 |
| Pb | 0.004 | 0.0013 | 0.00065 | 0.00091 |
| F | — | — | — | — |
| As | 0.01 | — | — | — |
| Se | 0.00005 | 0.00124 | 0.00086 | 0.000049 |
| Hg | — | — | — | — |
| CN | — | — | — | — |
| Cr | — | — | — | — |
| Cd | — | — | — | — |
| Al | 0.63 | 0.49 | 0.47 | 0.14 |

From Table 1, it was confirmed that the seawater showed a distinct difference from fresh water in the trace element items of vanadium, germanium and selenium depending on where the seawater was taken.

The seawater used for the preparation of each of Brassica oleracea var. acephala, Nasturtium officinale and Beta vulgaris root extracts was obtained by additionally filtering Seawater 2 of Table 1 through a 0.2 μm filter to remove impurities and then desalinating by electrodialysis (ED) and used.

### [Preparation Example 1] Preparation of Brassica oleracea Var. Acephala Seawater Extract

After cutting Brassica oleracea var. acephala into an appropriate size, pulverized product of Brassica oleracea var. acephala was prepared using a grinder, and then 20 g of the pulverized product was added to 0.5 L of seawater and immersed therein at about 15° C. for about 5 hours and subsequently filtered to obtain a Brassica oleracea var. acephala seawater extract (filtrate).

### [Preparation Example 2] Preparation of Nasturtium officinale Seawater Extract

After cutting Nasturtium officinale into an appropriate size, pulverized product of Nasturtium officinale was prepared using a grinder, and then 20 g of the pulverized product was added to 0.5 L of seawater and immersed therein at about 15° C. for about 5 hours and subsequently filtered to obtain a Nasturtium officinale seawater extract (filtrate).

### [Preparation Example 3] Preparation of Beta vulgaris Root Seawater Extract

After cutting Beta vulgaris root into an appropriate size, pulverized product of Beta vulgaris root was prepared using a grinder, and then 20 g of the pulverized product was added to 0.5 L of seawater and immersed therein at about 15° C. for about 5 hours and subsequently filtered to obtain a Beta vulgaris root seawater extract (filtrate).

### [Comparative Preparation Example 1] Preparation of Brassica Oleracea Var. Acephala Water Extract

After cutting Brassica oleracea var. acephala into an appropriate size, pulverized product of Brassica oleracea var. acephala was prepared using a grinder, and then 20 g of the pulverized product was added to 0.5 L of purified water and immersed therein at about 15° C. for about 5 hours and subsequently filtered to obtain a Brassica oleracea var. acephala water extract (filtrate).

### [Comparative Preparation Example 2] Preparation of Nasturtium officinale Water Extract

After cutting Nasturtium officinale into an appropriate size, pulverized product of Nasturtium officinale was prepared using a grinder, and then 20 g of the pulverized product was added to 0.5 L of purified water and immersed therein at about 15° C. for about 5 hours and subsequently filtered to obtain a Nasturtium officinale water extract (filtrate).

### [Comparative Preparation Example 3] Preparation of Beta vulgaris Root Water Extract

After cutting Beta vulgaris root into an appropriate size, pulverized product of Beta vulgaris root was prepared using a grinder, and then 20 g of the pulverized product was added to 0.5 L of purified water and immersed therein at about 15° C. for about 5 hours and subsequently filtered to obtain a Beta vulgaris root water extract (filtrate).

### [Example 1] Mixture of Brassica oleracea Var. Acephala Seawater Extract, Nasturtium officinale Seawater Extract and Beta vulgaris Root Seawater Extract

The Brassica oleracea var. acephala seawater extract, Nasturtium officinale seawater extract and Beta vulgaris root seawater extract were mixed in a weight ratio of 1:1:10 using Preparation Examples 1 to 3 to prepare a mixture.

### [Comparative Example 1] Mixture of Brassica oleracea Var. Acephala Water Extract, Nasturtium officinale Water Extract and Beta vulgaris Root Water Extract

The Brassica oleracea var. acephala water extract, Nasturtium officinale water extract and Beta vulgaris root water extract were mixed in a weight ratio of 1:1:10 using Comparative Preparation Examples 1 to 3 to prepare a mixture.

### <Test Example 1: Evaluation of Effect of Enhancing Skin Moisturizing Function of Mixture Consisting of Brassica oleracea Var. Acephala Seawater Extract, Nasturtium officinale Seawater Extract and Beta vulgaris Root Seawater Extract>

In order to evaluate the effect of enhancing the skin moisturizing function of the mixture sample of Brassica oleracea var. acephala seawater extract, Nasturtium officinale seawater extract and Beta vulgaris root seawater extract of Example 1, the mRNA expression levels of hyaluronan synthase 2 (HAS2), which produces hyaluronic acid, a major moisturizing factor for skin, and filaggrin, which is involved in the synthesis of natural moisturizing factors of keratinocytes were confirmed (see, Papakonstantinou et al., Dermatoendocrinol., 4(3):253-8, 2012; and Rawlings et al., Dermatol Ther., 17, Suppl 1:43-8, 2004).

Specifically, the mixture sample consisting of Brassica oleracea var. acephala seawater extract, Nasturtium officinale seawater extract and Beta vulgaris root seawater extract of Example land the mixture sample consisting of Brassica oleracea var. acephala water extract, Nasturtium officinale water extract and Beta vulgaris root water extract of Comparative Example 1 were diluted to a treatment concentration of 0.15% or 0.3% based on the volume and treated to keratinocytes. Then, the mRNA expression levels of HAS2 and filaggrin of keratinocytes were measured so as to compare the effect of enhancing the skin moisturizing function of Example 1 and

### Comparative Example 1

Measurement Method of HAS2 and Filaggrin Expression Level:

HEKn cells (human epidermal keratinocytes, neonatal) were seeded at a concentration of 2×10⁵ cells/dish in a 60 mm cell culture dish using KBM-Gold Keratinocyte Basal Medium (Lonza #192151) supplemented with KGM-Gold keratinocyte Growth Medium SingleQuot (Lonza #192152)) and then cultured for about 24 hours until confluency of about 80% was achieved in a 37° C., 5% CO₂ incubator. The cultured HEKn cells were with treated with the sample of Example 1 or the sample of Comparative Example 1 at a concentration of 0.3% (v/v) (i.e., adding 90 μL of the sample of Example 1 or Comparative Example 1 to a 60 mm cell culture dish containing 2910 μL of cell culture medium), and then incubated for about 24 hours. Thereafter, the medium used for the culture of HEKn cells was removed, and 1 ml of Trizol (Invitrogen) was added to isolate RNA according to the RNA isolation method of Invitrogen. The isolated RNA was quantified at 260 nm using a UV detector (Hewlett Packard), and then reverse-transcription was performed to synthesize cDNA, and real-time PCR (Real-Time Polymerase Chain Reaction) was performed to measure the mRNA expression levels of HAS2 and Filaggrin involved in TJ formation in keratinocytes. Here, in order to analyze the relative expression pattern for each sample, corrections were made based on the mRNA expression level of RPL13A (Ribosomal Protein L13a).

As a result, as shown in FIG. 1, it was confirmed that Example 1 (mixture consisting of Brassica oleracea var. acephala seawater extract, Nasturtium officinale seawater extract and Beta vulgaris root seawater extract showed significantly higher mRNA expression levels of HAS2 and filaggrin as compared to Comparative Example 1 (mixture consisting of Brassica oleracea var. acephala water extract, Nasturtium officinale water extract and Beta vulgaris root water extract).

### <Test Example 2: Evaluation of Effect of Enhancing Barrier Function of Skin of Mixture Consisting of Brassica oleracea Var. Acephala Seawater Extract, Nasturtium officinale Seawater Extract and Beta vulgaris Root Seawater Extract>

In order to evaluate the effect of enhancing the barrier function of the skin of the mixture sample of Example 1 consisting of Brassica oleracea var. acephala seawater extract, Nasturtium officinale seawater extract and Beta vulgaris root seawater extract, the mRNA expression level of occludin (OCLN), which is involved in the formation of tight junction (TJ) was confirmed.

Specifically, the mixture sample consisting of Brassica oleracea var. acephala seawater extract, Nasturtium officinale seawater extract and Beta vulgaris root seawater extract of Example 1 and the mixture sample consisting of Brassica oleracea var. acephala water extract, Nasturtium officinale water extract and Beta vulgaris root water extract of Comparative Example 1 were diluted to a treatment concentration of 0.15% or 0.3% based on the volume and treated to keratinocytes. Then, the mRNA expression level of OCLN of keratinocytes was measured so as to compare the effect of enhancing the barrier function of the skin of Example 1 and Comparative Example 1.

Measurement Method of OCLN Expression Level:

HEKn cells (human epidermal keratinocytes, neonatal) were seeded at a concentration of 2×10⁵ cells/dish in a 60 mm cell culture dish using KBM-Gold Keratinocyte Basal Medium (Lonza #192151) supplemented with KGM-Gold keratinocyte Growth Medium SingleQuot (Lonza #192152)) and then cultured for about 24 hours until confluency of about 80% was achieved in a 37° C., 5% CO₂ incubator. The cultured HEKn cells were with treated with the sample of Example 1 or the sample of Comparative Example 1 at a concentration of 0.15% (v/v) or 0.3% (v/v), and then incubated for about 24 hours. Thereafter, the medium used for the culture of HEKn cells was removed, and Trizol (Invitrogen) was added to isolate RNA according to the RNA isolation method of Invitrogen. The isolated RNA was quantified at 260 nm using a UV detector (Hewlett Packard), and then reverse-transcription was performed to synthesize cDNA, and real-time PCR (Real-Time Polymerase Chain Reaction) was performed to measure the mRNA expression level of OCLN involved in TJ formation in keratinocytes. Here, in order to analyze the relative expression pattern for each sample, corrections were made based on the mRNA expression level of RPL13A (Ribosomal Protein L13a).

As a result, as shown in FIG. 2, it was confirmed that (i) Example 1 (mixture consisting of Brassica oleracea var. acephala seawater extract, Nasturtium officinale seawater extract and Beta vulgaris root seawater extract) showed a significantly higher mRNA expression level of OCLN as compared to Comparative Example 1 (mixture consisting of Brassica oleracea var. acephala water extract, Nasturtium officinale water extract and Beta vulgaris root water extract) and (ii) in the case of Example 1, the mRNA expression level of OCLN was significantly higher at the treatment concentration of 0.3% (v/v) as compared to the treatment concentration of 0.15% (v/v).

In conclusion, the mixture of Brassica oleracea var. acephala seawater extract, Nasturtium officinale seawater extract and Beta vulgaris root seawater extract according to the present disclosure enhanced the skin moisturizing function by significantly increasing the expression levels of HAS2, filaggrin and OCLN and exhibited the effect of enhancing the barrier function of the skin by promoting the TJ formation. In particular, it was confirmed that the enhancement of the barrier function of the skin had an effect of increasing the skin moisturizing ability by reducing water loss of the skin.

Meanwhile, it is known that skin aging occurs due to a decrease in the content of extracellular matrix components such as collagen, elastin and hyaluronic acid present in the skin. To this end, it has been reported that skin aging is accelerated (decrease in skin elasticity, etc.) when it is accompanied by a decrease in water content of skin, and it has been found that CLDN (Claudin)-based proteins and OCLN, which are involved in formation of tight junction (TJ), are also involved in skin regeneration. Therefore, it was judged that the mixture of Brassica oleracea var. acephala seawater extract, Nasturtium officinale seawater extract and Beta vulgaris root seawater extract according to the present disclosure may exhibit anti-aging effects on the skin by enhancing the barrier function of the function, preventing skin water loss, and promoting skin cell turnover (skin regeneration, wound healing, etc.) (see, Bazzoni et al., J Cell Biol., 156(6):947-9. 2002; Furuse et al., J Cell Biol., 156(6):1099-111, 2002; Nakajima et al., J Pharmacol Exp Ther., 354(3):440-7., 2015; Volksdorf et al., Am J Pathol., 187(6):1301-1312, 2017; Papakonstantinou et al., Dermatoendocrinol., 4(3):253-8, 2012).

The cosmetic composition including the mixture of Brassica oleracea var. acephala seawater extract, Nasturtium officinale seawater extract and Beta vulgaris root seawater extract as an active ingredient according to the present disclosure can be prepared into various formulations (cosmetics, etc.) and can be controlled to a suitable content ratio in consideration of the functionality, cost and other conditions of the product to be implemented.

### [Formulation Example 1] Preparation of Ointment

An ointment including the mixture of Brassica oleracea var. acephala seawater extract, Nasturtium officinale seawater extract and Beta vulgaris root seawater extract according to the present disclosure was prepared by mixing the ingredients shown in Table 2 below including the oil-phase ingredients and aqueous-phase ingredients.

TABLE 2

| Mixing Ingredients | Content (wt %) |
|---|---|
| Purified water | Residual amount |
| Mixture of Brassica oleracea | 10.0 |
| var. acephala seawater extract, | |
| Nasturtium officinale | |
| seawater extract and | |
| Beta vulgaris | |
| root seawater extract | |
| Caprine/Capryl triglyceride | 10.0 |
| Liquid paraffin | 10.0 |
| Sorbitan sesquioleate | 6.0 |
| Octyldodeseth-25 | 9.0 |
| Cetyl ethylhexanoate | 10.0 |
| Squalane | 1.0 |
| Salicylic acid | 1.0 |
| Glycerin | 15.0 |
| Sorbitol | 10.0 |

### [Formulation Example 2] Preparation of Nourishing Cosmetic Water (Milk Lotion)

A nourishing cosmetic water including the mixture of Brassica oleracea var. acephala seawater extract, Nasturtium officinale seawater extract and Beta vulgaris root seawater extract according to the present disclosure was prepared by mixing the ingredients shown in Table 3 below including the oil-phase ingredients and aqueous-phase ingredients.

TABLE 3

| Mixing Ingredients | Content (wt %) |
|---|---|
| Purified water | Residual amount |
| Mixture of Brassica oleracea | 0.1 |
| var. acephala seawater extract, | |
| Nasturtium officinale | |
| seawater extract and | |
| Beta vulgaris | |
| root seawater extract | |
| Beewax | 4.0 |
| Polysorbate 60 | 1.5 |
| Sorbitan sesquioleate | 1.5 |
| Liquid paraffin | 0.5 |
| Montana 202 | 5.0 |
| (Manufacturer: Seppic) | |
| Glycerin | 3.0 |
| Butylene glycol | 3.0 |
| Propylene glycol | 3.0 |
| Carboxyvinyl polymer | 0.1 |
| Triethanolamine | 0.2 |
| Preservatives, pigments, flavors | q.s. |

### [Formulation Example 3] Preparation of Massage Cream

A massage cream including the mixture of Brassica oleracea var. acephala seawater extract, Nasturtium officinale seawater extract and Beta vulgaris root seawater extract according to the present disclosure was prepared by mixing the ingredients shown in Table 4 below including the oil-phase ingredients and aqueous-phase ingredients.

TABLE 4

| Mixing Ingredients | Content (wt %) |
|---|---|
| Purified water | Residual amount |
| Mixture of Brassica oleracea | 0.1 |
| var. acephala seawater extract, | |
| Nasturtium officinale | |
| seawater extract and | |
| Beta vulgaris | |
| root seawater extract | |
| Beewax | 10.0 |
| Polysorbate 60 | 1.5 |
| PEG-60 Hydrogenated castor oil | 2.0 |
| Sorbitan sesquioleate | 0.8 |
| Liquid paraffin | 40.0 |
| Squalane | 5.0 |
| Montana 202 | 4.0 |
| (Manufacturer: Seppic) | |
| Glycerin | 5.0 |
| Butylene glycol | 3.0 |
| Propylene glycol | 3.0 |
| Triethanolamine | 0.2 |
| Preservatives, pigments, flavors | q.s. |

### [Formulation Example 4] Preparation of Pack

A pack including the mixture of Brassica oleracea var. acephala seawater extract, Nasturtium officinale seawater extract and Beta vulgaris root seawater extract according to the present disclosure was prepared by mixing the ingredients shown in Table 5 below including the oil-phase ingredients and aqueous-phase ingredients.

TABLE 5

| Mixing Ingredients | Content (wt %) |
|---|---|
| Purified water | Residual amount |
| Mixture of Brassica oleracea | 0.1 |
| var. acephala seawater extract, | |
| Nasturtium officinale | |
| seawater extract and | |
| Beta vulgaris | |
| root seawater extract | |
| Polyvinyl alcohol | 13.0 |
| Sodium carboxymethylcellulose | 0.2 |
| Glycerin | 5.0 |
| Allantoin | 0.1 |
| Ethanol | 6.0 |
| PEG-12 nonylphenyl ether | 0.3 |
| Polysorbate 60 | 0.3 |
| Preservatives, pigments, flavors | q.s. |

Although specific parts of the present disclosure have been described in detail, it will be apparent to one of ordinary skilled in the art that these specific techniques are merely a preferred embodiment and that the scope of the present disclosure is not limited thereto. Therefore, the substantial scope of the present disclosure will be defined by the accompanying claims and their equivalents.

1. A method for enhancing the barrier function of the skin by applying onto the skin a cosmetic composition comprising: a mixture of Brassica oleracea var. acephala seawater extract, Nasturtium officinale seawater extract and Beta vulgaris root seawater extract as an active ingredient.

2. A method for improving skin moisturizing ability by applying onto the skin a cosmetic composition comprising: a mixture of Brassica oleracea var. acephala seawater extract, Nasturtium officinale seawater extract and Beta vulgaris Root seawater extract, as an active ingredient.

3. A method for promoting skin cell turnover by applying onto the skin a cosmetic composition comprising: a mixture of Brassica oleracea var. acephala seawater extract, Nasturtium officinale seawater extract and Beta vulgaris Root seawater extract, as an active ingredient.

4. The method of claim 3, wherein the skin cell turnover is achieved by keratinocyte turnover.

5. The method of claim 1, wherein the mixture is composed of Brassica oleracea var. acephala seawater extract, Nasturtium officinale seawater extract and Beta vulgaris root seawater extract in a mixing ratio of 1:1:3˜50.

6. The method of claim 1, wherein the mixture is contained in an amount of 0.001 to 40% by weight based on the total weight of the composition.

7. The method of claim 1, wherein the composition increases the expression level of a gene encoding hyaluronan synthase 2 (HAS2) of skin cells.

8. The method of claim 1, wherein the composition increases the expression level of a gene encoding filaggrin of skin cells.

9. The method of claim 1, wherein the composition increases the expression level of a gene encoding occludin (OCLN) of skin cells.

10. (canceled)

11. (canceled)

12. (canceled)

13. The method of claim 2, wherein the mixture is composed of Brassica oleracea var. acephala seawater extract, Nasturtium officinale seawater extract and Beta vulgaris root seawater extract in a mixing ratio of 1:1:3˜50.

14. The method of claim 2, wherein the mixture is contained in an amount of 0.001 to 40% by weight based on the total weight of the composition.

15. The method of claim 2, wherein the composition increases the expression level of a gene encoding hyaluronan synthase 2 (HAS2) of skin cells.

16. The method of claim 2, wherein the composition increases the expression level of a gene encoding filaggrin of skin cells.

17. The method of claim 2, wherein the composition increases the expression level of a gene encoding occludin (OCLN) of skin cells.

18. The method of claim 3, wherein the mixture is composed of Brassica oleracea var. acephala seawater extract, Nasturtium officinale seawater extract and Beta vulgaris root seawater extract in a mixing ratio of 1:1:3˜50.

19. The method of claim 3, wherein the mixture is contained in an amount of 0.001 to 40% by weight based on the total weight of the composition.

20. The method of claim 3, wherein the composition increases the expression level of a gene encoding hyaluronan synthase 2 (HAS2) of skin cells.

21. The method of claim 3, wherein the composition increases the expression level of a gene encoding filaggrin of skin cells.

22. The method of claim 3, wherein the composition increases the expression level of a gene encoding occludin (OCLN) of skin cells.
